(19) United States
(12) Patent Application Publication
(10) Pub. No.: US 2013/0327902 A1
(43) Pub. Date: Dec. 12, 2013

(54) STAND

(71) Applicant: Leica Microsystems (Schweiz) AG, Heerbrugg (CH)

(72) Inventors: Roman FRICK, Speicher (CH); Marco SCHUTZ, Rorschach (CH)

(21) Appl. No.: 13/909,497

(22) Filed: Jun. 4, 2013

(30) Foreign Application Priority Data
Jun. 6, 2012 (DE) 10 2012 209 594.2

(51) Int. Cl. A61B 19/00

(52) U.S. Cl. CPC A61B 19/26
USPC 248/122.1

(57) ABSTRACT

A stand for carrying a load, in particular a surgical microscope, having a first number N of pivot bearings, pivotable at pivot axes (120a-120g), for making available a pivotability of a number of arms (130a-130e) of the stand, and/or of a mount (140) provided on an arm for the load to be carried, relative to one another, such that in order to lock in place a desired pivot position, the pivot bearings can be acted upon at least in part by means of lockable and releasable brakes (125a-125h), where as a result of a first actuation of an actuation device (160, 162) a number n of pivot bearings, such that n≦N, is releasable; and as a result of a second actuation of the actuation device (160, 162) a number n′ of the pivot bearings, such that n′<n, is re-lockable.

### CROSS REFERENCE TO RELATED APPLICATIONS

This application claims priority of German patent application number 10 2012 209 594.2 filed Jun. 6, 2012, the entire disclosure of which is incorporated by reference herein.

### FIELD OF THE INVENTION

The present invention relates to a stand for carrying a load, in particular a surgical microscope, having a first number N of pivot bearings, pivotable at respective pivot axes, for making available a pivotability of a number of arms of the stand, and/or of a mount provided on one of the arms for mounting the load to be carried, relative to one another, such that in order to lock in place a desired pivot position the pivot bearings can be acted upon at least in part by means of lockable and releasable brakes.

### BACKGROUND OF THE INVENTION

It is essential for surgical microscopes that they be positionable and orientable in space as simply, precisely, and freely as possible. Surgical microscopes are mounted for this purpose on stands that permit such positionability and orientability.

Stands for surgical microscopes typically comprise a basic stand element that carries a number of stand arms articulatedly connected to one another. A carrier for a surgical microscope that is to be carried is provided at that end of the stand arms which is remote from the basic stand element.

By moving pivot bearings that connect the respective arms to one another, it is possible to position and orient the stand arms, and thus the surgical microscope, as desired in space, in which context the optical circumstances of the microscope can also be taken into account.

Such stands are known, for example, from DE 103 53 961 B4.

In order to lock in place a desired position or orientation of a surgical microscope, the pivot bearings of the stand are conventionally provided with electromagnetic brakes or with mechanical brakes.

When electromagnetic brakes are provided, it is usual that all brakes can be released simultaneously when a button is pressed. This is intended to ensure rapid actuation of the surgical microscope during a surgical procedure, for purposes of establishing and retaining a new position. Stands are also usually provided with displaceable compensation masses that are provided displaceably in order to balance out the stand as a function of the weight of a respectively used surgical microscope and/or of a specific position of the stand arms.

When electromagnetic brakes are used, it must be noted that uncontrolled or unexpected motions of the microscope can occur when the brakes are released with the stand in an unbalanced state. On the other hand, stands embodied with such electromagnetic brakes can as a rule be operated more smoothly and thus more comfortably for a surgeon.

Stands having mechanical brakes have the disadvantage that they operate more stiffly, which makes a surgeon's work overall more stressful.

### SUMMARY OF THE INVENTION

The present invention addresses the object of embodying a stand, embodied with brakes, for a surgical microscope in as easily operable a fashion as possible.

This object is achieved by a stand wherein as a result of a first actuation of an actuation device a number n of pivot bearings, such that n≦N (the number of pivot bearings), is releasable; and as a result of a second actuation of the actuation device a number n′ of the pivot bearings, such that n′<n, is re-lockable.

The stand according to the present invention makes possible particularly simple and flexible positioning and/or orientation of a surgical microscope in space. In particular, it is still possible in the context of the invention, upon a first actuation of an actuation device, to release all the brakes of the stand at once in convenient fashion in order to instantaneously make available maximum flexibility for a repositioning of the surgical microscope; but upon a subsequent second actuation for locking the brakes, to keep a predefined group of brakes released or open, thereby continuing to ensure partial pivotability of the stand so that, for example, a fine adjustment can still be carried out manually. The stand according to the present invention is advantageously provided as, for example, a stand displaceable on a foot or as a wall stand or ceiling stand.

Advantageous embodiments of the stand according to the present invention are the subject matter of the dependent claims.

According to the present invention it is possible, with a first actuation of the actuation apparatus, to release not the entire number of brakes but instead only a subset. As mentioned, however, it is preferred to release all the pivot bearings or brakes with a first actuation of the actuation member. This permits particularly convenient actuation of the stand.

Usefully, the brakes respectively associated with the pivot bearings comprise at least in part an electromagnetic brake unit and a friction brake unit. This ensures that after an only partial locking of the brakes, the brakes that remain open are manually actuatable, with the result that a fine adjustment of the corresponding stand arms remains possible. It is possible in particular to adjust the friction brake units individually, i.e. to make available a stronger or weaker frictional force in the respective units.

It is preferred that after a re-locking of a subset of the brakes, a pivot bearing adjacent to the load or to the surgical microscope continues to remain (manually) pivotable. This makes fine positioning of the microscope possible in simple fashion even if the remaining brakes or pivot bearings of the stand are locked or immobilized. This is advantageous in particular in that a surgeon does not need to actuate the actuation device for small shifts of the surgical microscope.

It is particularly preferred that different functions for releasing and/or locking pivot bearings or brakes are triggerable depending on the duration over time and/or location of an actuation of the actuation device. In particular, provision can also be made for releasing or locking only individual brakes by means of a corresponding actuation of the actuation device, or for successively releasing or locking individual brakes in a defined sequence by repeated actuation.

It is preferred in particular that the actuation device be actuatable by pressing or by swiping or swiping motions. The actuation device can thus be provided in the form of buttons, switches, or also as a keypad or touchscreen.

The stand according to the present invention usefully comprises a computer unit for computational selection in the context of the actuation of brakes that are to be locked or released. A computing device of this kind can be acted upon in particular by corresponding sensor signals from sensors that, for example sense torques and/or forces acting at various pivot bearings. A computer device of this kind can also be embodied to authorize a corresponding actuation of compensation weights of the stand. Usefully, the computer device is operable by means of a touchscreen or a keypad.

It is particularly preferred that the actuation device be embodied integratedly in a handle by means of which a user can grasp the stand for manual pivoting. Particularly ergonomic handling of a surgical microscope is obtained especially when the actuation device is embodied with a single button for acting upon the brakes.

Further advantages and embodiments of the invention are evident from the description and the appended drawings.

It is understood that the features recited above and those yet to be explained below are usable not only in the respective combination indicated, but also in other combinations or in isolation, without departing from the scope of the present invention.

### BRIEF DESCRIPTION OF THE DRAWING VIEW

The invention is schematically depicted in the drawings on the basis of an exemplifying embodiment, and will be described in detail below with reference to the drawings.

FIG. 1 is a schematic side view of a first preferred embodiment of the stand according to the present invention.

### DETAILED DESCRIPTION OF THE INVENTION

A preferred embodiment of the stand according to the present invention is designated in its entirety as 100.

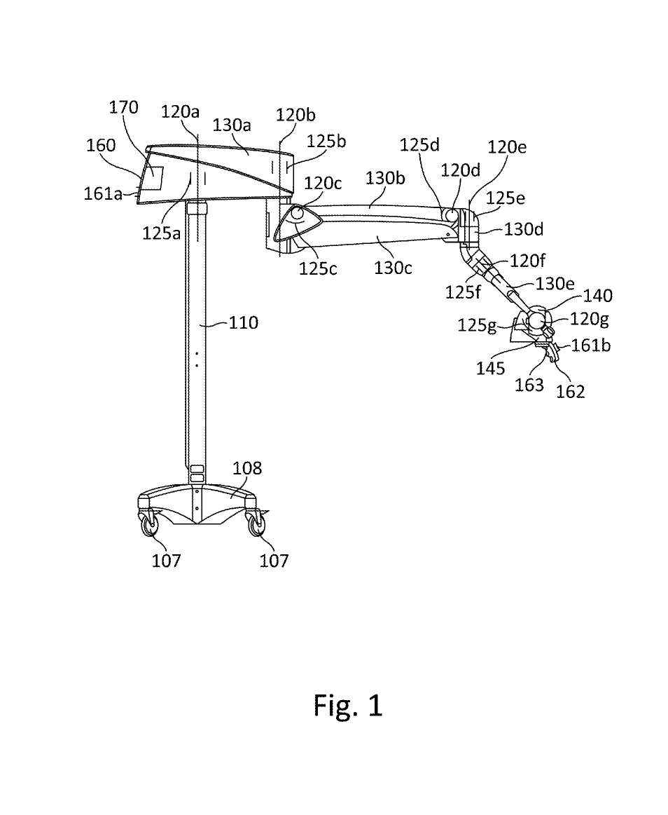

Stand 100 comprises a basic stand element (stand column) 110, mounted on a foot 108 embodied with casters 107, on which stand arms 130a, 130b, 130c, 130d, 130e are mounted pivotably around pivot bearings having respective pivot axes 120a, 120b, 120c, 120d, 120e, 120f, 120g. The pivot bearings are depicted here schematically by means of their respective pivot axes. It is evident that pivot axes 120a, 120b, 120e are provided substantially vertically, and pivot axes 120c, 120d, 120g are provided substantially horizontally. Pivot axis 120f is provided to extend at an inclination. Stand arms 120b, 120c are provided as parallel-arm links, as is apparent from FIG. 1.

A schematically depicted mount 140 for a surgical microscope 145 (likewise depicted schematically) is embodied at the outer end of pivot arm 130e, i.e. at that end of stand 100 which is opposite with respect to basic stand part 110. Mount 140 (and thus surgical microscope 145) is pivotable around the pivot bearing having pivot axis 120g.

Stand 100 serves for spatial positioning and orientation of surgical microscope 145.

At least one compensation mass (not depicted) can be provided in order to compensate for moments (torque moments) that act on stand 100 because of the weight of surgical microscope 145. To allow compensation for different surgical microscopes having correspondingly different weights, the compensation mass is usefully provided in a displaceable manner. It is, however, also possible to omit such compensation masses, in which case, for example, foot 108 can have a correspondingly large mass. Be it noted that instead of the foot, the stand according to the present invention can also be provided with a wall mount or as a wall stand, or with a ceiling mount as a ceiling stand. In these two cases a basic stand element or corresponding stand column 110 is not necessary.

The pivot bearings can be at least in part locked or immobilized in their respective pivot positions by means of correspondingly provided brakes. These brakes are depicted schematically and are labeled 125a to 125g. These brakes usefully each comprise, once again at least in part, an electromagnetic brake and a mechanical brake or friction brake. Be it noted that not every pivot bearing needs to be embodied with a brake. In the exemplifying embodiment depicted it is particularly preferred to provide exclusively the pivot bearings having pivot axes 120a, 120b, 120e, 120f, and 120g with brakes.

The stand further comprises an actuation device with which brakes 125a to 125g can be locked and released, as will be additionally described below. An actuation device of this kind can be provided at different positions of the stand; FIG. 1 depicts, by way of example, two actuation devices respectively labeled 160 and 162.

The stand further comprises a computer device 170 which can serve, for example, to sense (by means of corresponding sensors that are not depicted) torques acting on the respective pivot bearings and to authorize or effect an immobilization or release of specific brakes, optionally accompanied by shifting of a compensation weight. As is evident from FIG. 1, computer device 170 can be provided in stand arm 130a, for example, behind actuation device 160 that is preferably embodied as a touchscreen.

Let it be assumed by way of example that all brakes 125a to 125g associated with the respective pivot bearings are released by means of a touchpad field 161a or button 161b provided on actuation device 160 or 162. Be it noted again that the pivot bearings do not all need to be provided with a corresponding brake in order to make available a functional stand.

In this state, in which all brakes 125a to 125g are released, a surgeon can bring surgical microscope 145 into a desired position or orientation, for example, by actuating a handle 163. A portion of brakes 125a to 125g are brought back into the engaged position by a second actuation of actuation device 160 or 162, for example by releasing or re-pressing button 161b or by a second actuation of the touchpad field.

In this context, for example, only brake 125g that is associated with pivot bearing 120g remains in an open state, so that surgical microscope 145 is still manually pivotable with respect to that pivot axis 120g. Brake 125g comprises, besides an electromagnetic brake unit, a mechanical friction brake. In the state depicted, this enables manual fine adjustment of the position of surgical microscope 145. After such a fine adjustment, brake 125g can be locked again by means of a further actuation of the actuation device, this further actuation having no influence on the remaining brakes which remain locked. It is also conceivable, however, to leave brake 125g in the opened state by a correspondingly stronger adjustment of the friction brake.

It is particularly preferred to provide actuation device 162 in integrated fashion in handle 163, and to bring about the above-described first and second actuation by means of a single button 161b provided on the handle. Such an actuation of a single button that is integrated into a handle for pivoting the surgical microscope proves to be particularly favorable ergonomically.

In addition to action upon individual brakes, as depicted, further groups of brakes can also be locked and/or released simultaneously with one another. This kind of function of the partial brake control system 160, 161 usefully can be selected via the actuation device and can be activated and deactivated via computer unit 170. For example, different fields that are associated with respective partial brake control actions can be provided on a touchscreen.

It proves to be particularly advantageous in the context of the stand depicted that even inexperienced users, or users not yet familiar with a specific system made up of a stand and surgical microscope, can quickly and safely carry out a microscope positioning action, since fine positioning of the surgical microscope can be carried out in a state in which a majority of the pivot bearings are locked because the respective brakes are closed.

A further functionality that is conceivable, for example, is that as a result of a first pressing of the touchpad field or button 161a, 161b, all brakes 125a to 125g are released; as a result of a second pressing, only a portion of the brakes are locked; and as a result of a third pressing, all brakes are once again locked. Such functionalities can also be selected or triggered, for example, by way of the duration in time of the actuation of button 161b or of touchpad field 161a.

Usefully, those brakes which at present are released or locked can be displayed on the actuation apparatus, in particular on a touchscreen. It is also possible to indicate which brakes can safely be released without causing the overall system of stand and load to go out of balance.

### PARTS LIST

100 Stand

107 Casters

108 Foot for stand

110 Basic stand element (stand column)

120a-120g Pivot axes of pivot bearings

125a-125g Brakes

130a-130e Stand arms

140 Mount

145 Surgical microscope

160, 162 Actuation device

161a Touchpad field of actuation device

161b Button of actuation device

163 Handle

170 Computer device

What is claimed is:

1. A stand for carrying a load, in particular a surgical microscope, having a first number N of pivot bearings, pivotable at pivot axes (120a-120g), for making available a pivotability of a number of arms (130a-130e) of the stand, and/or of a mount (140) provided on an arm for the load to be carried, relative to one another, such that in order to lock in place a desired pivot position the pivot bearings can be acted upon at least in part by means of lockable and releasable brakes (125a-125h),
wherein as a result of a first actuation of an actuation device (160, 162) a number n of pivot bearings, such that n≦N, is releasable; and as a result of a second actuation of the actuation device (160, 162) a number n′ of the pivot bearings, such that n′<n, is re-lockable.

2. The stand according to claim 1, wherein n is equal to N.

3. The stand according to claim 1, wherein at least a portion of the brakes (125a-125g) have an electromagnetic brake unit and a friction brake unit.

4. The stand according to claim 1, wherein after a re-locking of the n′ pivot bearings, a pivot bearing that is adjacent to the load and is pivotable about an axis (120g) remains pivotable.

5. The stand according to claim 1, wherein depending on the length and/or location of an actuation of the actuation device (160, 162), different functions for releasing and/or locking pivot bearings or brakes (125a-125g) associated with them are triggerable.

6. The stand according to claim 1, wherein the actuation device is actuatable by pressing or swiping, in particular of buttons or switches or of a touchscreen or a keypad.

7. The stand according to claim 1, characterized by a computer device (170) for computational selection of pivot bearings to be locked and to be released.

8. The stand according to claim 1, wherein the actuation device (162) is embodied integratedly in a handle (163) by means of which a user can grasp the stand for manual pivoting.
